(19) United States
(12) Patent Application Publication
(10) Pub. No.: US 2007/0248692 A1
(43) Pub. Date: Oct. 25, 2007

(54) COMBINATION THERAPY USING PENTAFLUOROBENZENESULFONAMIDES AND ANTINEOPLASTIC AGENTS

(75) Inventors: Fengzhi Li, Buffala, NY (US); Xiang Ling, Snyder, NY (US); Youcef Rustum, East Amherst, NY (US); Susan Schwendner, Berkeley, CA (US); Pieter Timmermans, Redwood City, CA (US); Jacqueline Walling, Carpenters (GB)

Correspondence Address: BANNER & WITCOFF, LTD., 1100 13th STREET, N.W., SUITE 1200, WASHINGTON, DC, 20005-4051, US

(73) Assignees: AMGEN INC., Thousand Oaks, CA (US); HEALTH RESEARCH, INC., Buffalo, NY (US)

(21) Appl. No.: 11/687,097

(22) Filed: Mar. 16, 2007

Related U.S. Application Data
(63) Continuation of application No. 10/997,784, filed on Nov. 23, 2004, now abandoned.
Application No. 10/997,784 is a continuation-in-part of application No. 10/052,905, filed on Nov. 2, 2001, now Pat. No. 6,822,001.
(60) Provisional application No. 60/245,878, filed on Nov. 3, 2000.

(51) Int. Cl. A61K 36/00; A61K 31/18; A61K 31/415; A61P 35/00; A61K 31/44; A61K 35/00

(52) U.S. Cl. 424/725; 424/780; 514/293; 514/404; 514/604

(57) ABSTRACT

Combination therapies are provided for the treatment of proliferative disorders which use a pentafluorobenzenesulfonamide of formula I and an antineoplastic agent.

### CROSS-REFERENCES TO RELATED APPLICATIONS

This application is a continuation-in-part of U.S. Ser. No. 10/977,364, filed Oct. 29, 2004, pending, which is a continuation of U.S. Ser. No. 10/052,905, filed Nov. 2, 2001, issued as U.S. Pat. No. 6,822,001 on Nov. 23, 2004, which claims the benefit of U.S. Ser. No. 60/245,878, filed Nov. 3, 2000, abandoned, the disclosures of each of which are hereby incorporated herein by reference. Also, this application is related in technology to co-pending application Ser. No. 09/627,041, filed Jul. 27, 2000.

### STATEMENT AS TO RIGHTS TO INVENTIONS MADE UNDER FEDERALLY SPONSORED RESEARCH AND DEVELOPMENT

Not applicable

### BACKGROUND OF THE INVENTION

1. Field of the Invention

The present invention relates to combinations of pentafluorobenzenesulfonamides and various other chemotherapeutic agents that are capable of inhibiting abnormal cell proliferation.

2. Background

Cancer is a generic name for a wide range of cellular malignancies characterized by unregulated growth, lack of differentiation, and the ability to invade local tissues and metastasize. These neoplastic malignancies affect, with various degrees of prevalence, every tissue and organ in the body. A multitude of therapeutic agents have been developed over the past few decades for the treatment of various types of cancer. The most commonly used types of anticancer agents include: DNA-alkylating agents (e.g., cyclophosphamide, ifosfamide), antimetabolites (e.g., methotrexate, a folate antagonist, and 5-fluorouracil, a pyrimidine antagonist), microtubule disrupters (e.g., vincristine, vinblastine, paclitaxel), DNA intercalators (e.g., doxorubicin, daunomycin), and hormone therapy (e.g., tamoxifen, flutamide). The ideal antineoplastic drug would kill cancer cells selectively, with a wide therapeutic index relative to its toxicity towards non-malignant cells. It would also retain its efficacy against malignant cells, even after prolonged exposure to the drug. Unfortunately, none of the current chemotherapies possess an ideal profile. Most possess very narrow therapeutic indexes and, in practically every instance, cancerous cells exposed to slightly sublethal concentrations of a chemotherapeutic agent will develop resistance to such an agent, and quite often cross-resistance to several other antineoplastic agents.

The development of new anticancer agents has given rise to new treatment regimens and new combinations that are proving more effective in combating this disease.

Accordingly, it is one object of the present invention to provide compositions which directly or indirectly are toxic to actively dividing cells and are useful in the treatment of cancer.

A further object of the present invention is to provide methods for killing actively proliferating cells, such as cancerous, bacterial, or epithelial cells, and treating all types of cancers, and generally proliferative conditions. A further object is to provide methods for treating other medical conditions characterized by the presence of rapidly proliferating cells, such as psoriasis and other skin disorders.

Additional objects, features and advantages will become apparent to those skilled in the art from the following description and claims.

### SUMMARY OF THE INVENTION

In one aspect, the present invention provides compositions useful for the treatment of cancer and other diseases associated with abnormal cell proliferation. The compositions comprise an antineoplastic agent, including but not limited to prodrugs thereof, pharmaceutically acceptable salts of these agents and a compound having the formula:

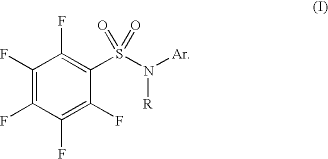

In the formula above, the letter R represents a hydrogen, substituted or unsubstituted (C₁-C₁₀)alkyl, or substituted or unsubstituted (C₃-C₆)alkenyl. The symbol Ar represents a substituted or unsubstituted aryl group or a substituted or unsubstituted heteroaryl group.

Suitable antineoplastic or antiproliferative agents include, but are not limited to, DNA-alkylating agents (e.g., cyclophosphamide, BCNU, busulfan and temozolamide), antimetabolites, antifolates and other inhibitors of DNA synthesis (e.g., methotrexate, 5-fluorouracil, gemcitabine), microtubule disruptors (e.g., vincristine, vinorelbine, paclitaxel, docetaxel), DNA intercalators (e.g., doxorubicin, daunomycin), hormone agents (e.g., tamoxifen, flutamide), topoisomerase I inhibitors (e.g., camptothecin analogs, including topotecan, camptothecin, CPT-11/irinotecan, and SN-38), topoisomerase II inhibitors (e.g., anthracyclines, epipodophyllotoxins, acridines, etoposide and teniposide) and DNA repair agents (e.g., hydroxyurea, camptothecin, etoposide), growth factor receptor kinase inhibitors (e.g., AG1478 and AG1296), biological response modifiers (including cytokines such as interferon α and growth factor inhibitors), antiangiogenic and antivascular agents (e.g., combretastatin A-4), antagonists of inhibitors of apoptosis (IAP) protein family members (e.g., c-IAP1, c-IAP2, X-chromosome-linked IAP (XIAP), mammalian IAP homolog A (MIHA), neuronal apoptosis inhibtor protein (NAIP), survivin, apollon, ML-IAP/livin and (IAP)-like protein-1 (ILP-1), (IAP)-like protein-2 (ILP-2), and other agents such as immunoconjugates (e.g., trasuzamab), antisense oligonucleotides and small interfering RNA (siRNA).

The compositions will, in some embodiments, contain a pharmaceutically acceptable carrier or diluent.

In another aspect, the present invention provides methods for the treatment of cancer and other proliferative disorders using the compositions provided above, or using the components in a sequential or simultaneous administration.

### BRIEF DESCRIPTION OF THE DRAWINGS

FIG. 1 is a graph which illustrates the effects of Compound 2 with gemcitabine in the treatment of MX-1 human mammary tumor xenografts in athymic nude mice, using suboptimal doses of each of the agents.

FIG. 2 is a graph which illustrates the effects of Compound 2 with paclitaxel in the treatment of MX-1 human mammary tumor xenografts in athymic nude mice, using suboptimal doses of each of the agents.

FIG. 3 provides the structures of Compound 1, Compound 2 and Compound 3.

FIG. 4 shows that inhibition of Compound 1-mediated survivin induction by siRNA-targeting survivin synergistically increased cell death. Each bar represents the mean±SD from three independent measurements.

FIG. 5 shows synergistic induction of cell death by Compound 1 in combination with a low concentration of SN-38 in MCF-7 cells. Each bar represents the means±SD from three independent measurements.

### DESCRIPTION OF THE SPECIFIC EMBODIMENTS

Definitions

The term “alkyl,” by itself or as part of another substituent, means, unless otherwise stated, a straight or branched chain, or cyclic hydrocarbon radical, or combination thereof, which may be fully saturated, mono- or polyunsaturated and can include di- and multivalent radicals, having the number of carbon atoms designated (i.e. C₁-C₁₀ means one to ten carbons). Examples of saturated hydrocarbon radicals include groups such as methyl, ethyl, n-propyl, isopropyl, n-butyl, t-butyl, isobutyl, sec-butyl, cyclohexyl, (cyclohexyl)methyl, cyclopropylmethyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. The term “alkyl,” unless otherwise noted, is also meant to include those derivatives of alkyl defined in more detail below as “heteroalkyl.” Alkyl groups which are limited to hydrocarbon groups are termed “homoalkyl”.

The term “alkylene” by itself or as part of another substituent means a divalent radical derived from an alkane, as exemplified by —CH₂CH₂CH₂CH₂—, and further includes those groups described below as “heteroalkylene.” Typically, an alkyl (or alkylene) group will have from 1 to 24 carbon atoms, with those groups having 10 or fewer carbon atoms being preferred in the present invention. A “lower alkyl” or “lower alkylene” is a shorter chain alkyl or alkylene group, generally having eight or fewer carbon atoms.

The terms “alkoxy,” “alkylamino” and “alkylthio” (or thioalkoxy) are used in their conventional sense, and refer to those alkyl groups attached to the remainder of the molecule via an oxygen atom, an amino group, or a sulfur atom, respectively.

The term “heteroalkyl,” by itself or in combination with another term, means, unless otherwise stated, a stable straight or branched chain, or cyclic hydrocarbon radical, or combinations thereof, consisting of the stated number of carbon atoms and from one to three heteroatoms selected from the group consisting of O, N, Si and S, and wherein the nitrogen and sulfur atoms may optionally be oxidized and the nitrogen heteroatom may optionally be quaternized. The heteroatom(s) O, N and S may be placed at any interior position of the heteroalkyl group. The heteroatom Si may be placed at any position of the heteroalkyl group, including the position at which the alkyl group is attached to the remainder of the molecule. Examples include —CH₂—CH₂—O—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CH—O—CH₃, —Si(CH₃)₃, —CH₂—CH═N—OCH₃, and —CH═CH—N(CH₃)—CH₃. Up to two heteroatoms may be consecutive, such as, for example, —CH₂—NH—OCH₃ and —CH₂—O—Si(CH₃)₃. Similarly, the term “heteroalkylene” by itself or as part of another substituent means a divalent radical derived from heteroalkyl, as exemplified by —CH₂—CH₂—S—CH₂CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied.

The terms “cycloalkyl” and “heterocycloalkyl”, by themselves or in combination with other terms, represent, unless otherwise stated, cyclic versions of “alkyl” and “heteroalkyl”, respectively. Additionally, for heterocycloalkyl, a heteroatom can occupy the position at which the heterocycle is attached to the remainder of the molecule. Examples of cycloalkyl include cyclopentyl, cyclohexyl, 1-cyclohexenyl, 3-cyclohexenyl, cycloheptyl, and the like. Examples of heterocycloalkyl include 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like.

The terms “halo” or “halogen,” by themselves or as part of another substituent, mean, unless otherwise stated, a fluorine, chlorine, bromine, or iodine atom. Additionally, terms such as “haloalkyl,” are meant to include monohaloalkyl and polyhaloalkyl. For example, the term “halo(C₁-C₄)alkyl” is meant to include trifluoromethyl, 2,2,2-trifluoroethyl, 4-chlorobutyl, 3-bromopropyl, and the like.

The term “aryl” means, unless otherwise stated, a polyunsaturated, typically aromatic, hydrocarbon substituent which can be a single ring or multiple rings (up to three rings) which are fused together or linked covalently. The term “heteroaryl” refers to aryl groups (or rings) that contain from zero to four heteroatoms selected from N, O, and S, wherein the nitrogen and sulfur atoms are optionally oxidized, and the nitrogen atom(s) are optionally quaternized. A heteroaryl group can be attached to the remainder of the molecule through a heteroatom. Non-limiting examples of aryl and heteroaryl groups include phenyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below.

For brevity, the term “aryl” when used in combination with other terms (e.g., aryloxy, arylthioxy, arylalkyl) includes both aryl and heteroaryl rings as defined above. Thus, the term “arylalkyl” is meant to include those radicals in which an aryl group is attached to an alkyl group (e.g., benzyl, phenethyl, pyridylmethyl and the like) including those alkyl groups in which a carbon atom (e.g., a methylene group) has been replaced by, for example, an oxygen atom (e.g., phenoxymethyl, 2-pyridyloxymethyl, 3-(1-naphthyloxy)propyl, and the like).

Each of the above terms (e.g., “alkyl,” “heteroalkyl,” “aryl” and “heteroaryl”) are meant to include both substituted and unsubstituted forms of the indicated radical. Preferred substituents for each type of radical are provided below.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) can be a variety of groups selected from: —OR′, ═O, ═NR′, ═N—OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NH—C(NH₂)═NH, —NR′C(NH₂)═NH, —NH—C(NH₂)═NR′, —S(O)R′, —S(O)₂R′, —S(O)₂NR′R″, —CN and —NO₂ in a number ranging from zero to (2m′+1), where m′ is the total number of carbon atoms in such radical. R′, R″ and R′″ each independently refer to hydrogen, unsubstituted (C₁-C₈)alkyl and heteroalkyl, unsubstituted aryl, aryl substituted with 1-3 halogens, unsubstituted alkyl, alkoxy or thioalkoxy groups, or aryl-(C₁-C₄)alkyl groups. When R′ and R″ are attached to the same nitrogen atom, they can be combined with the nitrogen atom to form a 5-, 6-, or 7-membered ring. For example, —NR′R″ is meant to include 1-pyrrolidinyl and 4-morpholinyl. From the above discussion of substituents, one of skill in the art will understand that the term “alkyl” is meant to include groups such as haloalkyl (e.g., —CF₃ and —CH₂CF₃) and acyl (e.g., —C(O)CH₃, —C(O)CF₃, —C(O)CH₂OCH₃, and the like).

Similarly, substituents for the aryl and heteroaryl groups are varied and are selected from: -halogen, —OR′, —OC(O)R′, —NR′R″, —SR′, —R′, —CN, —NO₂, —CO₂R′, —CONR′R″, —C(O)R′, —OC(O)NR′R″, —NR″C(O)R′, —NR″C(O)₂R′, —NR′—C(O)NR″R′″, —NH—C(NH₂)═NH, —NR′C(NH₂)═NH, —NH—C(NH₂)═NR′, —S(O)R′, —S(O)₂R′, —S(O)₂NR′R″, —N₃, —CH(Ph)₂, perfluoro(C₁-C₄)alkoxy, and perfluoro(C₁-C₄)alkyl, in a number ranging from zero to the total number of open valences on the aromatic ring system; and where R′, R″ and R′″ are independently selected from hydrogen, (C₁-C₈)alkyl and heteroalkyl, unsubstituted aryl and heteroaryl, (unsubstituted aryl)-(C₁-C₄)alkyl, and (unsubstituted aryl)oxy-(C₁-C₄)alkyl.

Two of the substituents on adjacent atoms of the aryl or heteroaryl ring may optionally be replaced with a substituent of the formula -T-C(O)—(CH₂)_{q}—U—, wherein T and U are independently —NH—, —O—, —CH₂— or a single bond, and q is an integer of from 0 to 2. Alternatively, two of the substituents on adjacent atoms of the aryl or heteroaryl ring may optionally be replaced with a substituent of the formula -A-(CH₂)ᵣ—B—, wherein A and B are independently —CH₂—, —O—, —NH—, —S—, —S(O)—, —S(O)₂—, —S(O)₂NR′— or a single bond, and r is an integer of from 1 to 3. One of the single bonds of the new ring so formed may optionally be replaced with a double bond. Alternatively, two of the substituents on adjacent atoms of the aryl or heteroaryl ring may optionally be replaced with a substituent of the formula —(CH₂)ₛ—X—(CH₂)ₜ—, where s and t are independently integers of from 0 to 3, and X is —O—, —NR′—, —S—, —S(O)—, —S(O)₂—, or —S(O)₂NR′—. The substituent R′ in —NR′— and —S(O)₂NR′— is selected from hydrogen or unsubstituted (C₁-C₆)alkyl.

As used herein, the term “heteroatom” is meant to include oxygen (O), nitrogen (N), sulfur (S) and silicon (Si).

The term “pharmaceutically acceptable salts” is meant to include salts of the active compounds which are prepared with relatively nontoxic acids or bases, depending on the particular substituents found on the compounds described herein. When compounds of the present invention contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present invention contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, oxalic, maleic, malonic, benzoic, succinic, suberic, fumaric, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, for example, Berge, S. M., et al, “Pharmaceutical Salts”, Journal of Pharmaceutical Science, 1977, 66, 1-19). Certain specific compounds of the present invention contain both basic and acidic functionalities that allow the compounds to be converted into either base or acid addition salts.

The neutral forms of the compounds may be regenerated by contacting the salt with a base or acid and isolating the parent compound in the conventional manner. The parent form of the compound differs from the various salt forms in certain physical properties, such as solubility in polar solvents, but otherwise the salts are equivalent to the parent form of the compound for the purposes of the present invention.

In addition to salt forms, the present invention provides compounds which are in a prodrug form. Prodrugs of the compounds described herein are those compounds that readily undergo chemical changes under physiological conditions to provide the compounds of the present invention. Additionally, prodrugs can be converted to the compounds of the present invention by chemical or biochemical methods in an ex vivo environment. For example, prodrugs can be slowly converted to the compounds of the present invention when placed in a transdermal patch reservoir with a suitable enzyme or chemical reagent.

Certain compounds of the present invention can exist in unsolvated forms as well as solvated forms, including hydrated forms. In general, the solvated forms are equivalent to unsolvated forms and are intended to be encompassed within the scope of the present invention. Certain compounds of the present invention may exist in multiple crystalline or amorphous forms. In general, all physical forms are equivalent for the uses contemplated by the present invention and are intended to be within the scope of the present invention.

Certain compounds of the present invention possess asymmetric carbon atoms (optical centers) or double bonds; the racemates, diastereomers, geometric isomers and individual isomers are all intended to be encompassed within the scope of the present invention.

The compounds of the present invention may also contain unnatural proportions of atomic isotopes at one or more of the atoms that constitute such compounds. For example, the compounds may be radiolabeled with radioactive isotopes, such as for example tritium (³H), iodine-125 (¹²⁵I) or carbon-14 (¹⁴C). All isotopic variations of the compounds of the present invention, whether radioactive or not, are intended to be encompassed within the scope of the present invention.

As used herein, a “camptothecin analog” or “camptothecin derivative” interchangeably refers to an antineoplastic agent comprising one or more substituents attached to the following core chemical structure: 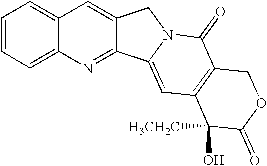
Suitable substituents include those provided herein for Ar (see, below). Additional substituents for camptothecin analogs are described in Goodman and Gilman's The Pharmacological Basis of Therapeutics, 10th Edition, 2001, pages 1422-1425, hereby incorporated herein by reference. Further camptothecin analogs and camptothecin derivatives, including suitable substituents for attaching to a camptothecin core molecule, are provided in U.S. Pat. Nos. 4,604,463; 5,004,758; 6,194,579; 6,207,832; 6,214,836; 6,218,399; 6,242,457; 6,310,210; 6,313,135; 6,376,617; 6,403,604; 6,436,951; 6,509,345; and 6,762,301. The terms “camptothecin analogs” or “camptothecin derivatives” further encompasses polymeric conjugates of camptothecin compounds, for example, those described in U.S. Pat. Nos. 5,773,522; 6,608,076 and in Bissett, et al. Br J Cancer (2004) 91:50; Oguma, et al., Biomed Chromatogr (2004) October 13; Yokoyama, et al. J Drug Target (2004) 12:373; Chirico, et al., Biophys Chem (2004) 110:281; and Barreiro-inglesias, et al., J Control Release (2004) 97:537; and Guiotto, et al., Bioorg Med Chem Lett (2004) 14:1803. All patents and publications described in the foregoing paragraph are hereby incorporated herein by reference.

As used herein, an inhibitor of apoptosis protein (IAP) family members includes those proteins which function to inhibit apoptosis and share the common structural motif termed a “baculorvirus IAP repeat” or BIR. A BIR is a zinc-binding motif consisting of a conserved sequence of about 70 amino acids (reviewed in Miller, Trends Cell Biol. (1999) 9:323). IAP family members and the BIR motif are reviewed in Salvesen and Duckett, Nat Rev Mol Cell Biol (2002) 3:401 and Reed, et al., Sci STKE (Jun. 22, 2004) 239:re9. All patents and publications described in the foregoing paragraph are hereby incorporated herein by reference.

As used herein, an inhibitor, or interchangeably, an antagonist of an inhibitor of apoptosis protein (IAP) family member includes any organic compound, amino acid sequence (i.e., peptide, peptidomimetic, peptoid), nucleic acid sequence (i.e., complementary DNA (cDNA), anti-sense RNA (asRNA), small inhibitory RNA (siRNA)) that inhibits the function of an IAP family member to inhibit or block apoptosis. An antagonist of an IAP family member can inhibit the expression (i.e., at the transcription or translation level) of an lAP family member protein or can inhibit the function of an IAP family member protein directly by binding to an IAP family member protein or indirectly by binding to a protein that is upstream or downstream from an IAP family member protein in a cellular signaling pathway. Inhibition of an IAP family member occurs when IAP family member-induced cellular apoptosis is inhibited at least 10%, 20%, 30%, 40%, 50%, 75% or 100% in the presence of an antagonist of an IAP family member, in comparison to IAP family member-induced cellular apoptosis in the absence of an antagonist of an IAP family member (i.e., a control).

General

A number of arylsulfonamides have recently been described for the treatment of disorders and conditions arising from abnormal cell proliferation and from elevated plasma cholesterol levels. See, for example, PCT publications WO 97/30677, WO 98/05315 and WO 99/10320. Representative of this new class of anticancer agents are the pentafluorobenzenesulfonamides described in WO 98/05315. These agents are thought to exert their effect by binding to β-tubulin and disrupting microtubule formation. See, Medina et al., Bioorganic & Med. Chem. Letters, 8(19):2653-56 (1998).

Still other pentafluorobenzenesulfonamides have been described in co-pending applications Ser. Nos. 60/090,681 filed Jun. 25, 1998 and 09/336,062 filed Jun. 18, 1999; Ser. Nos. 60/093,570 filed Jul. 20, 1998 and 09/353,976 filed Jul. 15, 1999; and Ser No. 60/100,888 filed Sep. 23, 1998.

Clinical trials are in progress to evaluate the pentafluorobenzene-sulfonamide class of compounds for the treatment of cancer, both alone and in combination with other agents. The concept of combination therapy is well exploited in current medical practice. Treatment of a pathology by combining two or more agents that target the same pathogen or biochemical pathway sometimes results in greater efficacy and diminished side effects relative to the use of the therapeutically relevant dose of each agent alone. In some cases, the efficacy of the drug combination is additive (the efficacy of the combination is approximately equal to the sum of the effects of each drug alone), but in other cases the effect can be synergistic (the efficacy of the combination is greater than the sum of the effects of each drug given alone). In real medical practice, it is often quite difficult to determine if drug combinations are additive or synergistic.

### DESCRIPTION OF THE EMBODIMENTS

Compositions

In one aspect, the present invention provides compositions comprising an antineoplastic agent and a compound having the formula: 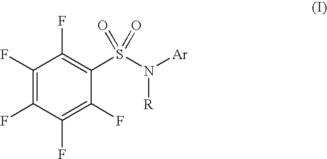
or a pharmaceutically acceptable salt thereof.

In the formula above, the letter R represents a hydrogen, substituted or unsubstituted (C₁-C₁₀)alkyl, or substituted or unsubstituted (C₃-C₆)alkenyl. The symbol Ar represents a substituted or unsubstituted aryl group or a substituted or unsubstituted heteroaryl group.

In preferred embodiments, R represents a hydrogen or a substituted or unsubstituted (C₁-C₄)alkyl group, more preferably hydrogen, methyl or ethyl.

Also preferred are those embodiments in which Ar represents a substituted aryl or substituted heteroaryl group, preferably those having a single ring (e.g., substituted phenyl, substituted pyridyl and substituted pyrimidyl). Particularly preferred embodiments are those in which Ar is substituted phenyl. For those embodiments in which Ar is substituted phenyl, the substituents will typically be present in a number of from one to three. Preferred substituents are selected from -halogen, —OR′, —OC(O)R′, —NR′R″, —SR′, —R′, —CN, —NO₂, —CO₂R′, —CONR′R″, —C(O)R′, —OC(O)NR′R″, —NR″C(O)R′, —NR″C(O)₂R′, —NR′—C(O)NR″R′″, —NH—C(NH₂)═NH, —NR′C(NH₂)═NH, —NH—C(NH₂)═NR′, perfluoro(C₁-C₄)alkoxy, and perfluoro(C₁-C₄)alkyl, where R′, R″ and R′″ are independently selected from hydrogen, (C₁-C₄)alkyl, unsubstituted aryl and heteroaryl, (unsubstituted aryl)-(C₁-C₄)alkyl, and (unsubstituted aryl)oxy-(C₁-C₄)alkyl. Particularly preferred substituents are halogen, (C₁-C₄)alkyl, —OR′, —OC(O)R′, —NR′R″, —CO₂R′, —CONR′R″, —C(O)R′, —OC(O)NR′R″, —NR″C(O)R′, —NR″C(O)₂R′, —NR′—C(O)NR″R′″, perfluoro(C₁-C₄)alkoxy, and perfluoro(C₁-C₄)alkyl, in which R′, R″ and R′″ are hydrogen or (C₁-C₄)alkyl. Still further preferred are those embodiments in which Ar is selected from:

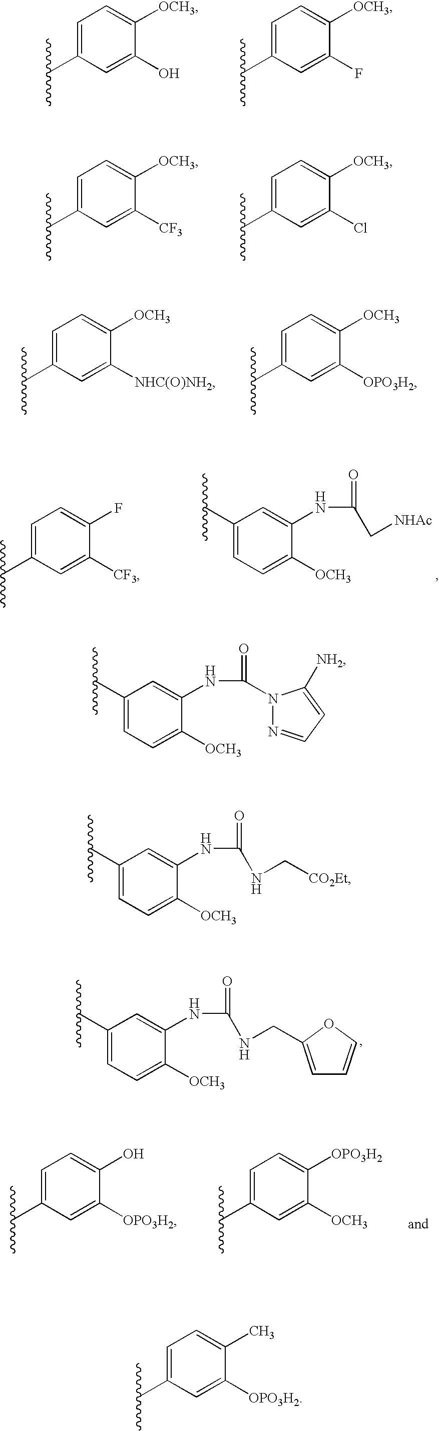

In the most preferred embodiments of the invention, the pentafluorobenzenesulfonamide compound used in the composition is selected from:

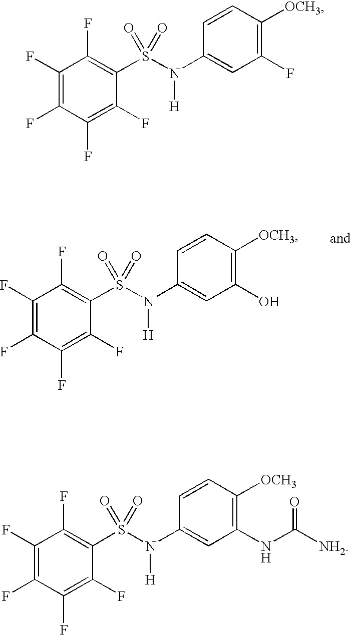

The compositions of the present invention will further comprise an antineoplastic agent. Suitable antineoplastic or antiproliferative agents include, but are not limited to, DNA-alkylating agents (e.g., cyclophosphamide, BCNU, busulfan and temozolamide), antimetabolites, antifolates and other inhibitors of DNA synthesis (e.g., methotrexate, 5-fluorouracil, gemcitabine), microtubule disruptors (e.g., vincristine, vinorelbine, paclitaxel, docetaxel), DNA intercalators (e.g., doxorubicin, daunomycin), hormone agents (e.g., tamoxifen, flutamide), topoisomerase I inhibitors (e.g., camptothecin analogs, including topotecan, camptothecin, CPT-11/irinotecan, and SN-38), topoisomerase II inhibitors (e.g., anthracyclines, epipodophyllotxoins, acridines, etoposide and teniposide) and DNA repair agents (e.g., hydroxyurea, camptothecin, etoposide), growth factor receptor kinase inhibitors (e.g., AG1478 and AG1296), biological response modifiers (including cytokines such as interferon α and growth factor inhibitors), antiangiogenic and antivascular agents (e.g., combretastatin A-4), and antagonists of inhibitors of apoptosis (IAP) protein family members (e.g., c-IAP1, c-IAP2, X-chromosome-linked IAP (XIAP), mammalian IAP homolog A (MIHA), neuronal apoptosis inhibtor protein (NAIP), survivin, apollon, ML-IAP/livin and (IAP)-like protein-1 (ILP-1), (IAP)-like protein-2 (ILP-2), and other agents such as immunoconjugates (e.g., trasuzamab), antisense oligonucleotides and small interfering RNA (siRNA).

Thus, in one embodiment of the present invention, the composition comprises a pentafluorobenzenesulfonamide as defined herein and an antineoplastic agent selected from the group consisting of DNA-alkylating agents, antimetabolites, antifolates and other inhibitors of DNA synthesis, microtubule disruptors, DNA intercalators, hormone agents, topoisomerase I/II inhibitors, DNA repair agents, growth factor receptor kinase inhibitors, biological response modifiers, antiangiogenic and antivascular agents, inhibitors of IAP family members, immunoconjugates, antisense oligonucleotides, and siRNA specific for one or more of an IAP family member nucleotide sequence.

In another embodiment, the composition comprises a pentafluorobenzenesulfonamide as defined herein and an antineoplastic agent selected from the group consisting of cyclophosphamide, BCNU (carmustine), busulfan, temozolomide, UFT, capecitabine, gemcitabine, cytarabine, improsulfan, piposulfan, benzodepa, carboquone, meturedepa, uredepa, altretamine, triethylenemelamine, triethylenephosphoramide, triethylenethiophosphoramide, trimethylolmelamine, chlorambucil, estramustine, ifosfamide, novembrichin, prednimustine, uracil mustard, dacarbazine, fluorouracil, methotrexate, mercaptopurine, thioguanine, vinblastine, vincristine, vinorelbine, vindesine, etoposide, teniposide, daunorubicin, doxorubicin, epirubicin, mitomycin, dactinomycin, daunomycin, plicamycin, bleomycin, L-asparaginase, camptothecin, hydroxyurea, procarbazine, mitotane, aminoglutethimide, tamoxifen, flutamide, mitoxantrone, paclitaxel, docetaxol, thiotepa, CPT-11/irinotecan, SN-38, and siRNA specific for one or more of an LAP family member nucleotide sequence, including one or more of a c-IAP1, c-IAP2, XIAP, MIHA, NAIP, survivin, apollon, ML-IAP/livin or ILP-2 nucleic acid sequence.

In preferred embodiments, the antineoplastic agent is gemcitabine or paclitaxel.

In preferred embodiments, the antineoplastic agent is a topoisomerase I inhibitor. In one preferred embodiment, the topoisomerase I inhibitor is a camptothecin analog. Exemplary camptothecin analogs include 7-ethyl-10-[4-(1-piperidino)-1-piperidino]carbonyloxycamptothecin (CPT-11/irinotecan) and its major oxidative metabolites, 7-ethyl-10-[4-N-(aminopentanoic acid)-1-piperidino]carbonyloxycamptothecin (APC) and 7-ethyl-10-[4-(1-piperidino)-1-amino]-carbonyloxycamptothecin (NPC) (see, Sanghani, et al., Drug Metab Dispos (2004) 32:505), 7-ethyl-10-hydroxy-camptothecin (SN-38), camptothecin, topotecan, 7-t-Butyldimethylsilyl-10-hydroxycamptothecin (DB67) (Lopez-Barcons, et al. Neoplasia (2004) 6:457), exatecan mesylate (DX-895 1f) (Clamp, et al. Gynecol Oncol (2004) 95:114), 9-nitrocamptothecin (RFS 2000 or rubitecan), 9-aminocamptothecin (IDEC-132), lurtotecan (OSI-211 or NX211 or GI-147211C), silatecan, gimatecan, homocamptothecins including diflomotecan (BN-80915) and BN-80927 (Demarquay, et al., Cancer Res (2004) 64:4942), and polymeric conjugates of camptothecin, including MAG-CPT (PNU 166148) (Bissett, et al., supra). Camptothecins are reviewed in Pommier Curr Med Chem Anti-Canc Agents (2004) 4:429; Zunino and Pratesi, Expert Opin Investig Drugs (2004) 13:269; Lansiaux and Bailly, Bull Cancer (2003) 90:239; and Ulukan and Swaan, Drugs (2002) 62:2039. All patents and publications described in the foregoing paragraph are hereby incorporated herein by reference.

In certain preferred embodiments the topoisomerase I inhibitor is a non-camptothecin topoisomerase I inhibitor. Exemplary non-camptothecin topoisomerase I inhibitors include indenoisoquinolines compounds (Xiao, et al, J Org Chem (2004) 69:7495), indolocarbazole compounds including NB-506 and [6-N-(1-hydroxymethyl-2-hydroxy)ethylamino-12,13-dihydro-2,10-dihydroxy-13-(beta-D-glucopyranosyl)-5H-indolo[2,3-a]-pyrrolo[3,4-c]-carbazole-5,7(6H)-dione] (J-107088 or edotecarin) (Yoshinari, et al., Cancer Res (1999) 59:4271), and tjipanazole analogues (Voldoire, et al., Bioorg Med Chem (2004) 12:1955). All patents and publications described in the foregoing paragraph are hereby incorporated herein by reference.

In certain preferred embodiments, the antineoplastic agent is a dual inhibitor of both topoisomerase I and topoisomerase II. Exemplary dual topoisomerase I and II inhibitors include triptycene analogs or “TT bisquinones” (Perchellet, et al. Anticancer Drugs (2004) 15:929), (N-[2-(dimethylamino)ethyl]acridine-4-carboxamide) (XR5000 or DACA), the benzopyridoindole intoplicine, the indenoquinolinone TAS-103, the benzophenazine XR11576, and the pyrazoloacridine NSC 366140 (reviewed in Denny and Baguley, Curr Top Med Chem (2003) 3:339, see also, de Jonge, et al. Br J Cancer (2004) 91:1459), tyrphostin derivatives (Bendetz-Nezer, et al., Mol Pharmacol (2004) 66:627), and F11782 (Kruczynski, et al., Clin Cancer Res (2004) 10:3156). All patents and publications described in the foregoing paragraph are hereby incorporated herein by reference.

In preferred embodiments, the antineoplastic agent is a direct or indirect antagonist of an inhibitor of apoptosis protein (IAP) family member. The IAP antagonist can be an organic chemical compound, a peptide, peptide mimetic or peptoid, an antisense RNA or small inhibitory RNA specific for a nucleic acid sequence of an IAP family member, or an antibody molecule that specifically binds to an LAP family member (e.g., humanized monoclonal antibody, a Fab fragment, single-chain antibody variable binding region (scFv)). Exemplary organic chemical compounds that are direct or indirect antagonists of an IAP family member include camptothecin analogs, including CPT-11/irinotecan and SN-38, polyphenylurea-based XIAP inhibitors (Wrzesien-Kus, et al. Apoptosis (2004) 9:705), XIAP antagonists described by Oost, et al. (J Med. Chem. (2004) 47:4417), flavopiridol (Rosato, et al. Leukemia (2004) 18:1780), and Bisphenol A diglycidyl ether (BADGE) (Fehlberg, et al. Br. J. Pharmacol. (2003) 139:495. Exemplary peptide, peptidomimetic, or peptoid antagonists of an IAP family member are described in U.S. Pat. No. 6,608,026, and US Patent Publications 2004/0171554, 2003/0073629, 2002/0177557, 2002/0160975, 2002/0132786. Exemplary IAP antisense nucleic acids, and anti-IAP antibodies are described in US Patent Publication 2002/0120121. All patents and publications described in the foregoing paragraph are hereby incorporated herein by reference.

In certain preferred embodiments, the antineoplastic agent is a dual inhibitor of a topoisomerase I enzyme and an IAP family member.

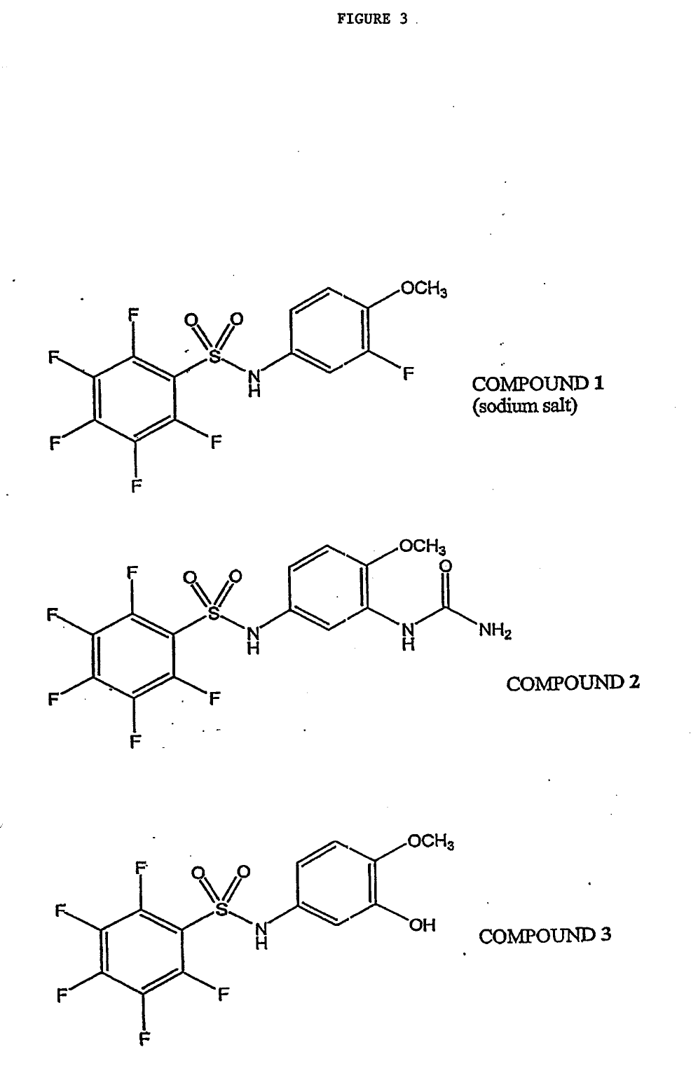

As noted above, in the most preferred embodiments of the present invention, the pentafluorobenzenesulfonamide compound used in the compositions is selected from Compound 1, Compound 2, and Compound 3 (see FIG. 3). While an understanding of the mechanism by which these compounds are metabolized is not necessary in order to practice the present invention, it is believed that glutathione conjugation plays a major role. Some preferred embodiments of the invention entail the use of compositions comprising Compound 1, Compound 2, or Compound 3 with an antineoplastic agent whose metabolism also is dependent, at least in part, on the formation of a glutathione conjugate (which may include, e.g., BCNU, cyclophosphamide, and thiotepa). Determination of glutathione metabolism can be accomplished according to standard methods known to those of skill in the art (see, e.g., Mannervik and Widersten in ADV. IN DRUG METAB. IN MAN, G. M. Pacifici and G. N. Fracchis, eds., European Commission, Luxemburg: 407-459 (1995), using glutathione transferases available from commercial sources such as PanVera, product nos. P2175, P2192 and P2177, and Research Diagnostics). Enhanced efficacy may be observed with such combinations due to competition for glutathione metabolism. Depending on the agents involved, this may result in depletion of glutathione levels, delayed metabolism of one or both agents, and increased exposure of the malignant tissue to one or more of the composition's active components.

Methods of Treating Proliferative Disorders

The present invention provides, in another aspect, methods for the treatment of proliferative disorders. In one embodiment, treatment is carried out using a composition comprising each of the two agents described above. In another embodiment, treatment comprises separate administration of one or more antineoplastic agents and a pentafluorophenylsulfonamide of formula I.

i. Combination Composition

In this embodiment of the invention, a composition of two or more agents (described above) is administered to a patient in need of treatment. The amount of each agent will typically be less than an amount that would produce a therapeutic effect if administered alone. The precise method of administration will depend on the patient and the judgment of the clinician, but will preferably be intravenous.

ii. Compositions Used Sequentially (Administer Each Separately)

In this embodiment of the invention, conventional protocols are described for the administration of an antineoplastic agent and compound 1 (as representative of the compounds of formula I). One of skill in the art will understand that various changes can be made by the clinician, depending on the particular agents selected for use and the routes and timing of administration. Thus, the present invention contemplates that the antineoplastic agent and the compounds of formula I can be administered sequentially on the same day, on concurrent days, or up to about 4 weeks apart.

The antineoplastic agent is preferably administered with a single intravenous infusion on day one of compound 1 administration period about four hours after the first day's administration of compound 1. To maintain sufficient hydration, one liter of normal saline with 20 meq KCl/L and 1 gm of magnesium sulfate, at a rate of about 250 ml/hour is administered prior to and after the infusion. Additional fluid may be given to maintain adequate urine output.

The treatment cycle may be continued until a clinical response is achieved or until intolerable side effects are encountered. The dosages of compound 1 and/or antineoplastic agent may be increased with each new treatment cycle, provided intolerable side effects are not encountered. The dosages may also be decreased if intolerable side effects are encountered. It is presently preferred to gradually adjust the dosage of compound 1 while holding the antineoplastic agent dosage constant.

As alluded to previously, certain preferred embodiments of the present invention entail combination therapy involving a pentafluorobenzenesulfonamide compound selected from Compound 1, Compound 2, and Compound 3 (see FIG. 3) and at least one other antineoplastic agent, wherein metabolism of the other antineoplastic agent(s) is dependent, at least in part, on the formation of a glutathione conjugate. In such embodiments, the order of administration may be especially important; that is, the order of administration may result in enhanced efficacy while minimizing adverse effects. In some preferred embodiments, it is preferable to administer Compound 1, Compound 2 or Compound 3 prior to the other antineoplastic agent, while in other preferred embodiments it is advantageous to co-administer the agents.

A common, but tolerable side effect of antineoplastic agent is nausea and vomiting. This can be alleviated by administering an anti-emetic (e.g., Ondansetron®, Granisetron®, Decadron®, Haldol®, Benadryl®, Ativan® and the like).

Of course, other forms of administration of both active ingredients, as they become available, are contemplated, such as by nasal spray, transdermally, by suppository, by sustained release dosage form, by IV injection, etc. Any form of administration will work so long as the proper dosages are delivered without destroying the active ingredient.

The effectiveness of treatment may be determined by controlled clinical trials. Patients having cancer with measurable or evaluable tumors will be included in the study. A measurable tumor is one that can be measured in at least two dimensions such as a lung tumor surrounded by aerated lung, a skin nodule, or a superficial lymph node. An evaluable tumor in one that can be measured in one dimension such as a lung tumor not completely surrounded by aerated lung or a palpable abdominal or soft tissue mass that can be measured in one dimension. Tumor markers which have been shown to be highly correlated with extent of disease will also be considered to provide an evaluable disease, such as PSA for prostate cancer, CA-125 for ovarian cancer, CA-15-3 for breast cancer, etc.

The tumor will be measured or evaluated before and after treatment by whatever means provides the most accurate measurement, such as CT scan, MRI scan, Ultrasonography, etc. New tumors or the lack thereof in previously irradiated fields can also be used to assess the anti-tumor response. The criteria for evaluating response will be similar to that of the WHO Handbook of Reporting Results of Cancer Treatment, WHO Offset Publication 1979, 49-World Health Organization, Geneva. The following results are defined for uni- and bi-dimensionally measurable tumors.

Complete response: Complete disappearance of all clinically detectable malignant disease determined by two observations not less than four weeks apart.

Partial Response: (a) for bidimensionally measurable tumors, a decrease of at least 50% in the sum of the products of the largest perpendicular diameters of all measurable tumors as determined by two observations not less than four weeks apart. (b) for unidimensionally measurable tumors, a decrease by at least 50% in the sum of the largest diameters of all tumors as determined by two observations not less than four weeks apart. In cases where the patient has multiple tumors, It is not necessary for all tumors to have regressed to achieve a partial response as defined herein, but no tumor should have progressed and no new tumor should appear.

Stable disease: (a) for bidimensionally measurable tumors, less than a 50% decrease to less than a 25% increase in the sum of the products of the largest perpendicular diameters of all measurable tumors. (b) for unidimensionally measurable tumors, less than a 50% decrease to less than a 25% increase in the sum of the diameters of all tumors. For (a) and (b) no new tumors should appear.

No clinical response, i.e. progressive disease in defined as an increase of more than 50% in the product of the largest perpendicular diameters for at least one bidimensionally measurable tumor, or an increase of more than 25% in measurable dimension of at least one unidimensionally measurable tumor.

Of course elimination or alleviation of other known signs or symptoms of cancer, especially those listed previously can also be used to evaluate the effectiveness of this invention.

The cancers should be evaluated, i.e. tumors measured, etc., no more than 14 days before the start of the treatment. These cancers should be reevaluated about 28 days after day 1 of administration of the first dose of compound 1 and antineoplastic agent. Twenty eight days after this initial administration another administration period may be performed, and evaluations performed 28 days after the start of this second cycle. The treatment cycles may be continued until a clinical response is achieved or unacceptable toxicity is encountered.

Another aspect of this invention is the treatment of cancer with reduced side effects normally associated with an antineoplastic agent. This objective can be achieved by administration of lower doses of the two active ingredients or by shorter duration of dosing brought about by the synergistic effect of the combination.

### EXAMPLES

### Example 1

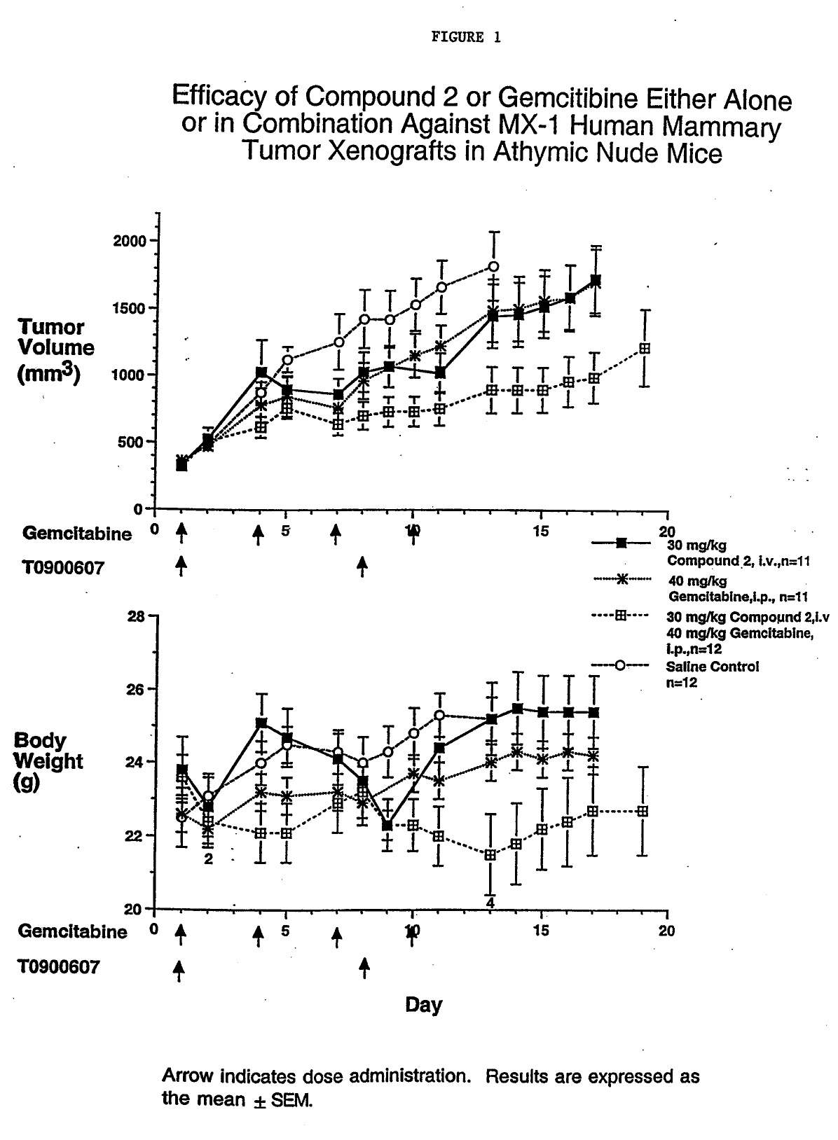

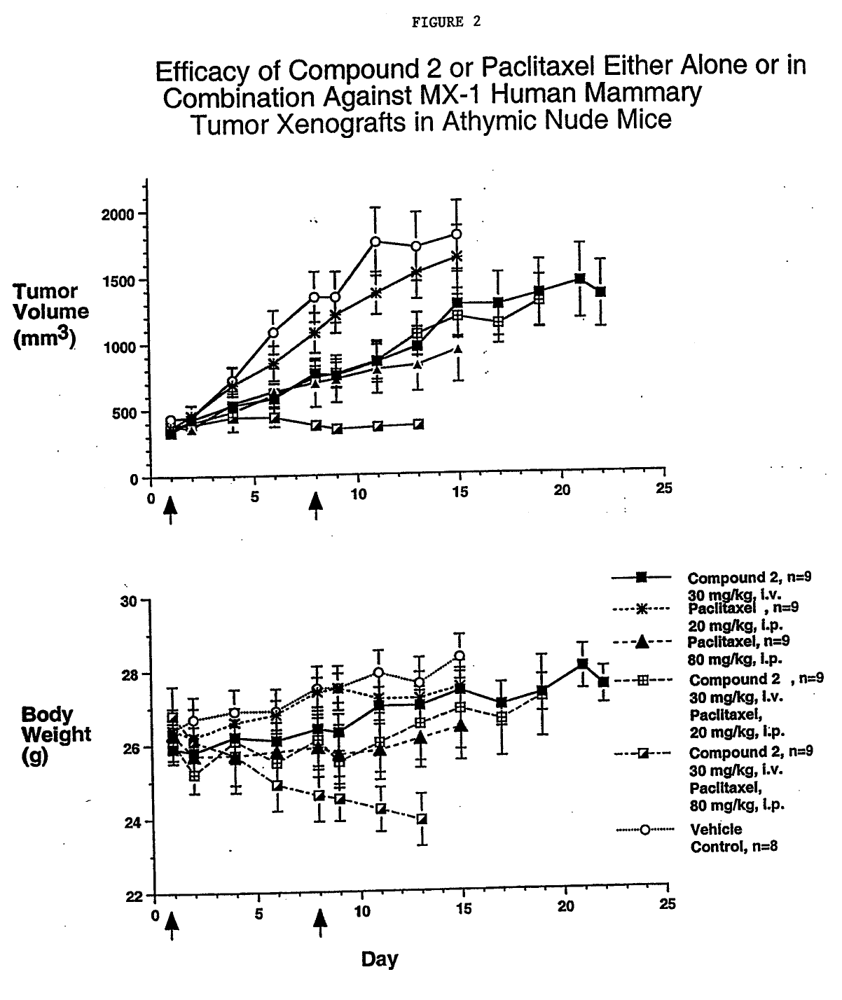

FIGS. 1 and 2 illustrate the effect achieved by combining a pentafluorobenzenesulfonamide with gemcitabine or with paclitaxel.

### Example 2

This example shows how combining a pentafluorobenzenesulfonamide with a topoisomerase I inhibitor synergistically promotes increased cell death of cancer cells. The exemplified topoisomerase inhibitor is SN-38, an active metabolite of irinotecan/CPT-11. Irinotecan/CPT-11 and SN-38 can also function as an antagonist of an IAP family member.

### Methods

Cell Culture

MCF-7 cells were maintained in RPMI 1640 and A549 lung cancer cells were maintained in DMEM. Both were supplemented with 10% fetal bovine serum (FBS) (MediaTech CellGro, Hemdon, Va.), penicillin (100 units/ml) and streptomycin (0.1 μg/ml) (Invitrogen Co., Grand Island, N.Y.) in a humidified atmosphere incubator with 5% CO2 at 37° C. Cells were routinely sub-cultured twice weekly.

Reagents

SN-38 was supplied by Pharmacia (Kalamazoo, Mich.). Anti-actin and peroxidase-conjugated goat anti-rabbit IgG were purchased from Sigma (St. Louis, Mo.). Anti-survivin (FL-142), anti-Erk (K-23) and anti-Akt (H-136) antibodies were purchased from Santa Cruz Biotechnology Co (Santa Cruz, Calif.). Phospho-Akt (Ser473) and phosphor-p44/42 MAP kinase (Thr202/Tyr204) antibodies were purchased from Cell Signaling Technology (Beverly, Mass.). Oligotransfectaminem reagent was purchased from Invitrogen (Carlsbad, Calif.).

Drug Dilution and Usage

Compound 1 stock solutions were 10 mM in DMSO and stored at −20° C. Just prior to Compound 1 treatment, the stock solution was freshly diluted to final concentration in growth medium. SN-38 was dissolved in DMSO to make a 5 mM stock solution, and the stock solution was diluted into final concentrations with growth medium. For all experiments, the control group was treated with the same amount of DMSO. Survivin expression was analyzed by Western blotting after treatment as described below. Cell morphologies with or without drug treatment were microscopically photographed under a inverted phase-contrast microscope. The percentage of dead cells was then determined by trypan blue exclusion assay as described below and the resultant data were plotted as a histogram.

Trypan Blue Exclusion Staining for Determination of Cell Viability

Cells to be counted were collected by trypsinization/centrifugation and resuspended in PBS buffer. A small sample of the cell suspension was diluted in 0.4% (w/v) trypan blue (one sample a time since viable cells absorb trypan blue over time as well). A cover glass was centered over the hemacytometer chambers and one chamber was filled with the cell dilution using a Pasteur pipette. Stained (dead) and unstained (viable) cells were counted in each of the four corner and central squares under an inverted microscope using 100× magnification, respectively. Each cell sample was counted in this way in triplicate. The percentage of cell viability or cell death in each sample was calculated.

Western Blot Analysis

Cells were washed with PBS and lysed on ice for 30 minutes in PBS containing 1% Nonidet P-40, 0.5% sodium deoxycholate, 0.1% sodium dodecyl sulfate (SDS), 10 μg/ml phenylmethyl sulfonyl fluoride, and 20 μM leupeptin. Cell lysates were clarified by centrifugation at 15,000×g for 20 minutes at 4° C. and the supernatant was used for Western blot analysis. Up to 75 μg of total protein from each sample was mixed in an equal volume of 2×SDS sample buffer and heated at 95° C. for 5 minutes, separated on 10-15% SDS-polyacrylamide gel electrophoresis (SDS-PAGE) gels, and electrotransferred on Immobilon-P membranes (Millipore, Bedford, Mass.) using semi-dry electrophoretic transfer apparatus. After the nonspecific binding sites on the membranes were blocked using 5% skimmed milk or bovine serum albumin (BSA) in TBS-T [20 mM Tris-HCl (pH 7.5), 0.137 M NaCl, and 0.01% Tween 20] for 3 h at room temperature with constant shaking, the membranes were incubated in TBS-T containing the relevant primary antibodies (1: 500-1000) and 5% BSA overnight at 4° C. with gentle shaking. After washing with TBS-T, the membrane was incubated in 5% skim milk in TBS-T buffer containing relevant second antibodies (1:5000) for 60 minutes at room temperature. Proteins were detected by a HRPL kit (National Diagnostics/LPS, Rochester, N.Y.) and visualized by autoradiography with various exposure times (20-120 seconds). For normalization of protein loading, the same membranes were stripped with stripping buffer (100 mM 2-mercaptoethanol, 2% sodium dodecyl sulphate, 62.5 mM Tris-HCl pH 6.7) and immunoblotted with a monoclonal antibody against actin or total kinase proteins at a dilution of 1:1000 by the same procedure.

SiRNA Preparation

Human survivin mRNA-specific RNA oligonucleotides with 3′-TT overhangs were chemically synthesized and purified by HPLC (Xeragon, Huntsville, Ala.): SRi-2f (⁹²GCG CCU GCA CCC CGG AGC G¹¹⁰TT) and SRi-2r (¹⁰⁰CGC UCC GGG GUG CAG GCG C⁹²TT). Equal moles of SRi-2f/SRi-2r (designated SRi-2) were mixed together to a final concentration of 20 μM in annealing buffer (100 mM KAc, 30 mM HEPES-KOH, 2 mM MgAc2, pH 7.4). After denaturation at 90° C. for 1 minute, the mixture (Sri-2) was annealed at 37° C. for 60 minutes and stored at −80° C. for transfection experiments. A scramble RNA duplex (designated scSRi) was also prepared as above for a negative control in this study. The scramble sequence [5′CAG UCG CGU UUG CGA CUG GTT (forward chain) and 5′CCA GUC GCA AAC GCG ACU GTT (reverse chain)] was not present in mammalian cells by BLAST search at NCBI.

In Vitro Transfection with siRNAs

Cells were transfected with survivin siRNAs using the Oligofectamine™ reagent (Invitrogen) following the manufacturer's instruction. Briefly, one day prior to transfection, 5×10⁴ MCF-7 cells per well were seeded in six-well plates (corresponding to a density of 40% at the time of transfection) without antibiotics. The transfection mixture was prepared by mixing 175 μl DMEM containing 6 μl 20 μM siRNA with 15 μl DMEM containing 3 μl Oligofectamine™ reagents. Before transfection, the medium in 6-well plates was replaced with serum-free DMEM medium (800 μl per well). The transfection mixture was added to the 6-well plate between 20-40 minutes after mixture preparation in a total volume of 990 μl per well. The transfected cells were incubated at 37° C. for 4 hours, and then 500 μl of DMEM medium containing 30% FBS was added. Cells were treated with and without Compound 124 hours after transfection with siRNA (SRi-2) or control siRNA (scSRi) as described above. All transfection experiments were performed in triplicate for each experiment. The cell morphology was photographed under an inverted phase-contrast microscope. The percentage of dead cells was then determined by trypan blue exclusion assay and plotted as a histogram.

### Results

Compound 1 Upregulates the Expression of Survivin without Affecting Bcl-2

It was determined whether modulation of survivin expression is involved in Compound 1 actin mechanism. Surprisingly, it was found that Compound 1 significantly upregulated survivin expression over the time tested in a dose-dependent manner in MCF-7 breast cancer cells. The surprise comes from the fact that Compound 1 was shown to be more efficacious against MDR (multidrug resistance) gene-overexpressed tumor cells in both cell culture and mice xenograft models than that of taxol and vinblastine (Shan et al., (1999) Proc. Natl. Acad. Sci. 96:5686). This finding indicated that the effects of Compound 1 in promoting cancer cell death could be synergized by the combined inhibition of survivin expression.

Compound 1 Treatment Increases the Phosphorylation of Akt and Erk1/2 but Decreases the Phosphorylation of p38 MAPK

In association with the upregulation of survivin in MCF-7 breast cancer cells after Compound 1 treatment, treatment of MCF-7 cells with Compound 1 increased the phosphorylation of Akt and Erk1/2 while total Akt and Erk1/2 did not change. In contrast, Compound 1 treatment of MCF-7 cells induced a decrease of p38 MAPK phosphorylation without affecting the total p38 protein.

Inhibition of Compound 1-induced Survivin Expression by Survivin siRNA Synergistically Induces Cell Death

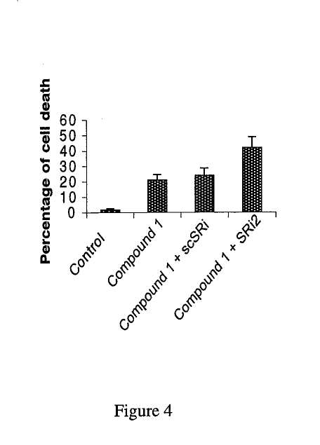

To determine whether inhibition of Compound 1-induced survivin expression could synergistically induce cell death, a previously tested survivin mRNA-specific siRNA (SRi-2) was transfected into MCF-7 cells 24 hours before Compound 1 treatment (Ling, et al., (2004) J. Biol. Chem. 279:15196). MCF-7 cells were plated in 12-well plates (10⁵ cells/well) and grown for 24 h. Cells were transfected with or without surviving siRNA (SRi-2) or scramble siRNA (scSRi) and treated 24 hours after transfection with or without Compound 1 (100 nM) for 24 h. Cell death was monitored by trypan blue exclusion as described in the Methods Section, and the resultant data were plotted as a histogram. Blocking Compound 1-induced survivin expression synergistically increased Compound 1-mediated cell death (FIG. 4).

SN-38, an Active Metabolite of CPT-11/Irinotecan, Down Regulates Survivin Expression

SN-38 down regulates survivin expression in breast cancer cells. Down regulation of survivin expression by SN-38 is more efficient at a low concentration than at a high concentration.

Inhibition of Survivin Expression by SN-38 Increases Phosphorylation of p38 MAP Kinase without Affecting the Phosphorylation of ErbB2

To determine whether SN-38-mediated down regulation of survivin is also associated with the activation/phosphorylation of p38 MAPK, MCF-7 breast cancer cells were treated with SN-38 and the phosphorylation of p38 were determined by Western blotting. SN-38 treatment increased the phosphorylation of p38 MAPK.

Compound 1 Treatment of MCF-7 Cells in Combination with a Low Concentration of SN-38 Synergistically Induces Cell Death

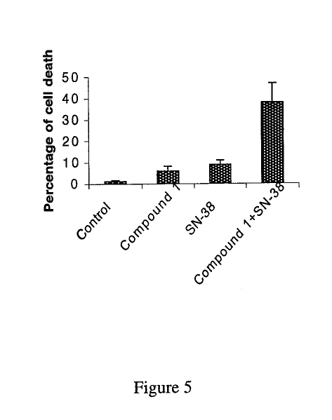

Compound 1 treatment of MCF-7 breast cancer cells in combination with a low concentration of SN-38 synergistically increased the effectiveness of Compound 1 in inducing cell death in comparison to the cell death obtained from either compound alone. Cells were plated in a 96 well plate (2000 cells/well) and grown for 24 h. Cells were sequentially treated with SN-38 for 16 hrs at 30 nM and then treated with or without Compound 1 (30 nM) for 48 h. Cell death was monitored by trypan blue exclusion. The resultant data were plotted in a histogram (FIG. 5).

All publications and patent applications cited in this specification are herein incorporated by reference as if each individual publication or patent application were specifically and individually indicated to be incorporated by reference. Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

What is claimed is:

1. A composition for the treatment of proliferative disorders, comprising an antineoplastic agent and a compound having the formula: 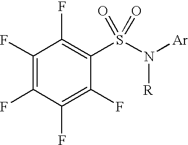
and pharmaceutically acceptable salts thereof;
wherein
R is a member selected from the group consisting of hydrogen and substituted or unsubstituted (C₁-C₁₀)alkyl; and
Ar is a member selected from the group consisting of substituted or unsubstituted aryl and substituted or unsubstituted heteroary

2. A composition in accordance with claim 1, wherein said antineoplastic agent is selected from the group consisting of DNA-alkylating agents, antimetabolites, antifolates and other inhibitors of DNA synthesis, microtubule disruptors, DNA intercalators, hormone agents, topoisomerase I/II inhibitors, DNA repair agents, growth factor receptor kinase inhibitors, biological response modifiers, antiangiogenic and antivascular agents, inhibitors of an IAP family member, immunoconjugates, antisense oligonucleotides and siRNA.

3. A composition in accordance with claim 1, wherein said antineoplastic agent is selected from the group consisting of cyclophosphamide, BCNU, busulfan, temozolomide, UFT, capecitabine, cytarabine, improsulfan, piposulfan, benzodepa, carboquone, meturedepa, uredepa, altretamine, triethylenemelamine, triethylenephosphoramide, triethylenethiophosphoramide, trimethylolmelamine, chlorambucil, estramustine, ifosfamide, novembrichin, prednimustine, uracil mustard, dacarbazine, fluorouracil, methotrexate, mercaptopurine, thioguanine, vinblastine, vincristine, vinorelbine, vindesine, etoposide, teniposide, daunorubicin, doxorubicin, epirubicin, mitomycin, dactinomycin, daunomycin, plicamycin, bleomycin, L-asparaginase, camptothecin, hydroxyurea, procarbazine, mitotane, aminoglutethimide, tamoxifen, flutamide, mitoxantrone, docetaxol, CPT/irinotecan, SN-38 and thiotepa.

4. A composition in accordance with claim 1, wherein said antineoplastic agent comprises at least one topoisomerase I inhibitor.

5. A composition in accordance with claim 4, wherein said topoisomerase inhibitor comprises a camptothecin analog.

6. A composition in accordance with claim 5, wherein said camptothecin analog is selected from the group consisting of CPT-11/irinotecan, SN-38, APC, NPC, camptothecin, topotecan, exatecan mesylate, 9-nitrocamptothecin, 9-aminocamptothecin, lurtotecan, silatecan, gimatecan, diflomotecan, BN-80927 and MAG-CPT, and mixtures thereof.

7. A composition in accordance with claim 1, wherein said antineoplastic agent comprises an antagonist of an IAP family member.

8. A composition in accordance with claim 1, wherein said antineoplastic agent is selected from the group consisting of doxorubicin, daunorubicin, CPT/irinotecan and SN-38.

9. A composition in accordance with claim 1, wherein said antineoplastic agent comprises CPT/irinotecan or SN-38.

10. A composition in accordance with claim 1, wherein R is hydrogen or unsubstituted (C₁-C₄)alkyl.

11. A composition in accordance with claim 1, wherein Ar is a substituted phenyl group.

12. A composition in accordance with claim 11, wherein said substituents on said phenyl group are selected from the group consisting of halogen, (C₁-C₄)alkoxy, (C₁-C₄)alkyl, —OPO₃H₂,

13. A composition in accordance with claim 12, wherein Ar represents a member selected from the group consisting of

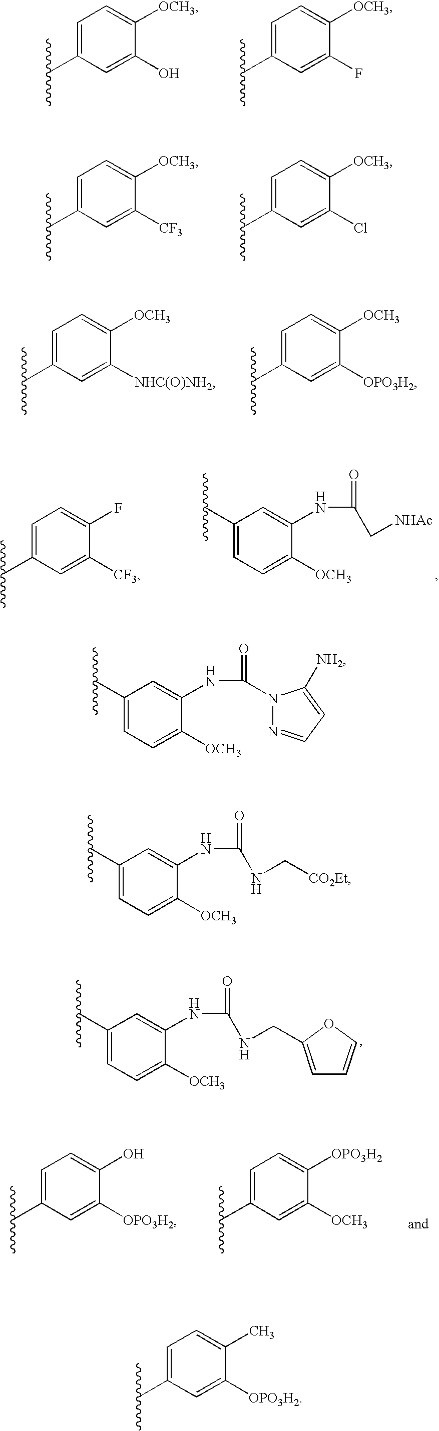

14. A composition in accordance with claim 1, wherein said compound is selected from the group consisting of:

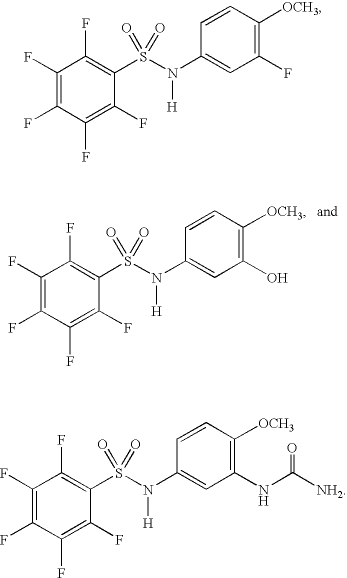

15. A method for the treatment of a proliferative disorder, comprising administering to a subject in need of such treatment an effective amount of a composition of claim 1.

16. A. method in accordance with claim 15, wherein said compound is selected from the group consisting of:

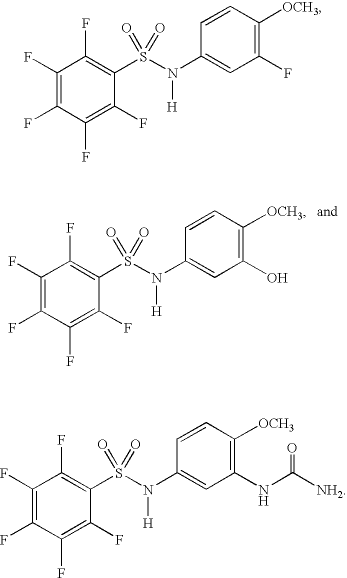

17. A method in accordance with claim 16, wherein said antineoplastic agent is selected from the group consisting of DNA-alkylating agents, antimetabolites, antifolates and other inhibitors of DNA synthesis, microtubule disruptors, DNA intercalators, hormone agents, topoisomerase I/II inhibitors, DNA repair agents, growth factor receptor kinase inhibitors, biological response modifiers, antiangiogenic and antivascular agents, immunoconjugates and antisense oligonucleotides.

18. A method in accordance with claim 16, wherein said antineoplastic agent is selected from the group consisting of cyclophosphamide, BCNU, busulfan, temozolomide, UFT, capecitabine, cytarabine, improsulfan, piposulfan, benzodepa, carboquone, meturedepa, uredepa, altretamine, triethylenemelamine, triethylenephosphoramide, triethylenethiophosphoramide, trimethylolmelamine, chlorambucil, estramustine, ifosfamide, novembrichin, prednimustine, uracil mustard, dacarbazine, fluorouracil, methotrexate, mercaptopurine, thioguanine, vinblastine, vincristine, vinorelbine, vindesine, etoposide, teniposide, daunorubicin, doxorubicin, epirubicin, mitomycin, dactinomycin, daunomycin, plicamycin, bleomycin, L-asparaginase, camptothecin, hydroxyurea, procarbazine, mitotane, aminoglutethimide, tamoxifen, flutamide, mitoxantrone, docetaxol, CPT/irinotecan, SN-38 and thiotepa.

19. A method in accordance with claim 16, wherein said antineoplastic agent comprises at least one topoisomerase I inhibitor.

20. A method in accordance with claim 19, wherein said topoisomerase I inhibitor comprises a camptothecin analog.

21. A method in accordance with claim 20, wherein said camptothecin analog is selected from the group consisting of CPT-11/irinotecan, SN-38, APC, NPC, camptothecin, topotecan, exatecan mesylate, 9-nitrocamptothecin, 9-aminocamptothecin, lurtotecan, silatecan, gimatecan, diflomotecan, BN-80927 and MAG-CPT, and mixtures thereof.

22. A method in accordance with claim 16, wherein said antineoplastic agent comprises an antagonist of an IAP family member.

23. A method in accordance with claim 16, wherein said antineoplastic agent is selected from the group consisting of doxorubicin, daunorubicin, CPT/irinotecan and SN-38.

24. A method in accordance with claim 16, wherein said antineoplastic agent comprises CPT/irinotecan or SN-38.

25. A method for the treatment of a proliferative disorder, comprising administering to a subject in need of such treatment:
i) a first amount of an antineoplastic agent; and
ii) a second amount of a compound of formula: 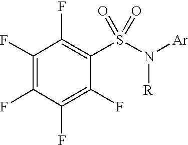
and pharmaceutically acceptable salts thereof; wherein
R is a member selected from the group consisting of hydrogen and substituted or unsubstituted (C₁-C₁₀)alkyl; and
Ar is a member selected from the group consisting of substituted or unsubstituted aryl and substituted or unsubstituted heteroaryl;
wherein said first amount and said second amount, in combination, are effective to treat said proliferative disorder

26. A method in accordance with claim 25, wherein said compound is selected from the group consisting of

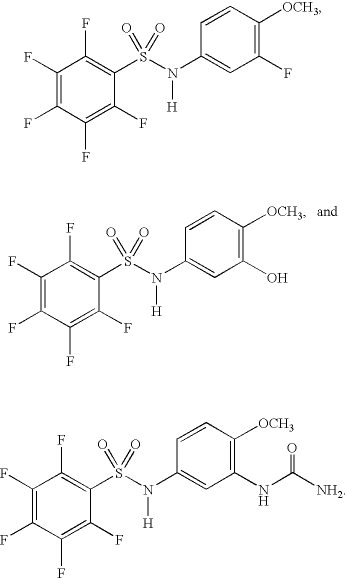

27. A method in accordance with claim 26, wherein said antineoplastic agent is selected from the group consisting of DNA-alkylating agents, antimetabolites, antifolates and other inhibitors of DNA synthesis, microtubule disruptors, DNA intercalators, hormone agents, topoisomerase I/II inhibitors, DNA repair agents, growth factor receptor kinase inhibitors, biological response modifiers, antiangiogenic and antivascular agents, immunoconjugates and antisense oligonucleotides.

28. A method in accordance with claim 26, wherein said antineoplastic agent is selected from the group consisting of cyclophosphamide, BCNU, busulfan, temozolomide, UFT, capecitabine, cytarabine, improsulfan, piposulfan, benzodepa, carboquone, meturedepa, uredepa, altretamine, triethylenemelamine, triethylenephosphoramide, triethylenethiophosphoramide, trimethylolmelamine, chlorambucil, estramustine, ifosfamide, novembrichin, prednimustine, uracil mustard, dacarbazine, fluorouracil, methotrexate, mercaptopurine, thioguanine, vinblastine, vincristine, vinorelbine, vindesine, etoposide, teniposide, daunorubicin, doxorubicin, epirubicin, mitomycin, dactinomycin, daunomycin, plicamycin, bleomycin, L-asparaginase, camptothecin, hydroxyurea, procarbazine, mitotane, aminoglutethimide, tamoxifen, flutamide, mitoxantrone, docetaxol, CPT/irinotecan, SN-38 and thiotepa.

29. A method in accordance with claim 26, wherein said antineoplastic agent comprises a topoisomerase I inhibitor.

30. A method in accordance with claim 29, wherein said topoisomerase I inhibitor comprises a camptothecin analog.

31. A method in accordance with claim 30, wherein said camptothecin analog is selected from the group consisting of CPT-11/irinotecan, SN-38, APC, NPC, camptothecin, topotecan, exatecan mesylate, 9-nitrocamptothecin, 9-aminocamptothecin, lurtotecan, silatecan, gimatecan, diflomotecan, BN-80927 and MAG-CPT, and mixtures thereof.

32. A method in accordance with claim 26, wherein said antineoplastic agent comprises an antagonist of an IAP family member.

33. A method in accordance with claim 26, wherein said antineoplastic agent is selected from the group consisting of doxorubicin, daunorubicin, CPT/irinotecan and SN-38.

34. A method in accordance with claim 26, wherein said antineoplastic agent is CPT/irinotecan or SN-38.

35. A method in accordance with claim 26, wherein said antineoplastic agent is administered prior to said compound.

36. A method in accordance with claim 26, wherein said antineoplastic agent is administered after said compound.

37. A method in accordance with claim 26, wherein said antineoplastic agent is administered simultaneously with said compound.


SEQUENCE LISTING (4 sequences)

SEQ ID NO: 1 - DNA, 21 bp, Artificial
  Si RNA related to human Survivin
gcgccugcaccccggagcgtt

SEQ ID NO: 2 - DNA, 21 bp, Artificial
  Si RNA related to human Survivin
cgcuccggggugcaggcgctt

SEQ ID NO: 3 - DNA, 21 bp, Artificial
  SiRNA scramble sequence
cagucgcguuugcgacuggtt

SEQ ID NO: 4 - DNA, 21 bp, Artificial
  SiRNA scramble sequence
ccagucgcaaacgcgacugtt